(19) United States
(12) Patent Application Publication
(10) Pub. No.: US 2024/0149061 A1
(43) Pub. Date: May 9, 2024

(54) SYSTEMS AND METHODS FOR TREATING PAIN AND/OR INFLAMMATION

(71) Applicant: Alivio Technologies, Inc., Coatesville, PA (US)

(72) Inventor: Caitlin WEST, Coatesville, PA (US)

(21) Appl. No.: 18/478,187

(22) Filed: Sep. 29, 2023

Related U.S. Application Data
(60) Provisional application No. 63/423,682, filed on Nov. 8, 2022.

(51) Int. Cl. A61N 1/36; A61N 2/00; A61N 2/02; A61N 5/067; G16H 20/30

(52) U.S. Cl. CPC A61N 1/36031; A61N 2/002; A61N 2/02; A61N 5/067; G16H 20/30; A61N 2005/0659

(57) ABSTRACT

Disclosed are techniques for treating pain and/or inflammation. In an aspect, a method of treating pain and/or inflammation includes determining, based on information associated with a treatment recipient, that a flare-up of pain and/or inflammation (“flare-up”) is in progress or that a likelihood of a flare-up exceeds a threshold level, and providing, to the treatment recipient, treatment to avoid or mitigate the flare-up. In some aspects, the treatment recipient can select a preferred treatment. In some aspects, the treatment is provided to the treatment recipient via a portable, wearable unit that includes hardware for providing multiple therapies, such heat, cold, infrared light, transcutaneous electrical nerve stimulation, and low frequency pulsed electromagnetic field, singly, in sequence, and/or in combination. In some aspects, a software application detects the flare-up or potential flare-up via a machine learning model and provides treatment via the portable, wearable unit.

### CROSS-REFERENCE TO RELATED APPLICATIONS

The present application for patent claims priority to U.S. Provisional Patent Application No. 63/423,682, entitled “SYSTEMS AND METHODS FOR TREATING PAIN AND INFLAMMATION ASSOCIATED WITH ARTHRITIS”, filed Nov. 8, 2022, which is assigned to the assignee hereof and expressly incorporated herein by reference in its entirety.

### BACKGROUND OF THE DISCLOSURE

### 1. Field of the Disclosure

Aspects of the disclosure relate generally to treatment of chronic pain and/or inflammation associated with arthritis and other ailments.

### 2. Description of the Related Art

Many individuals across the United States and around the world suffer from chronic pain, including chronic pain and/or inflammation caused by a degenerative autoimmune disease, Psoriatic Arthritis (PsA). Even with health insurance, the cost of therapeutic infusions (i.e., IV administered medication) and other treatments (e.g., prescriptions and injections for pain) can be very high. For example, one infusion, even with health insurance coverage, can cost $5,000 dollars. And even in such instances, the infusion may only be administered every eight weeks. Often, the medication is only productive for six weeks, resulting in two weeks of pain and/or inflammation flare-ups. Those suffering from symptoms may be left bedridden or limited in their ability to move. Not only are joint flare-ups painful, but those suffering symptoms may experience swelling or “puffing up” of their skin, making the flare-ups visible to others. Those who are fortunate enough to receive treatments are looking for ways to prolong the effects of the medicine. The average infusion is three hours long and can cost up to $20,000. At home injections are painful and can cost up to $2,000 even with insurance. Both treatment methods are critical for the treatment of arthritis but often come with the debilitating side effects of inflammation, pain, and stiffness.

There is an abundant need for chronic pain and/or inflammation relief as 57.5M (18%) of the U.S. population suffer from arthritis. Of these, 25.7M adults are limited in their daily activities due to their arthritis and this number is expected to grow to 35M by 2040. It is estimated that about 1.5M people have Psoriatic arthritis (“PsA”). PsA is a chronic, inflammatory disease of the joints where tendons and ligaments connect to bone. The diagnosis is made based on a doctor's observations and often the symptoms are similar to rheumatoid arthritis, gout, and other reactive arthritis. Often, the key indicator is if the patient has Psoriasis and is experiencing significant joint pain then he or she is diagnosed with PsA. PsA develops in 30% of the U.S. population that currently suffers from Psoriasis, 7.5M U.S. citizens, with flare-ups beginning between the ages of 10 and 20 years old. Psoriasis tends to progress to PsA when patients are between the ages of 30 and 50. There is no cure for PsA and even though there is a growing number of treatments, most are expensive and time intensive. Moreover, chronic inflammation compounds over time, resulting in severe joint damage and medical complications in the future.

As used herein, the terms “user”, “person”, “patient”, and “treatment recipient” may be used interchangeably unless otherwise noted. It will be understood, however, that a person receiving treatment therapies is a treatment recipient whether or not that person is a patient of a medical provider. I will be further understood that a person using a system, apparatus, or device to provide treatment therapies for themselves is a user of that system. It will be further understood that a method, apparatus, or device may determine a state of a person whether or not that person is a user of that method, apparatus, or device, e.g., when a user's condition is detected based on analysis of data provided by the user even when the user is not using that method, apparatus, or device.

### SUMMARY

The following presents a simplified summary relating to one or more aspects disclosed herein. Thus, the following summary should not be considered an extensive overview relating to all contemplated aspects, nor should the following summary be considered to identify key or critical elements relating to all contemplated aspects or to delineate the scope associated with any particular aspect. Accordingly, the following summary has the sole purpose to present certain concepts relating to one or more aspects relating to the mechanisms disclosed herein in a simplified form to precede the detailed description presented below.

In an aspect, a method of treating pain and/or inflammation includes determining, based on information associated with a treatment recipient, that a flare-up of pain and/or inflammation is in progress or that a likelihood of a flare-up of pain and/or inflammation exceeds a threshold level; and providing, to the treatment recipient, treatment to avoid or mitigate the flare-up of pain and/or inflammation.

In an aspect, an apparatus for treating pain and/or inflammation includes at least one memory; at least one wireless transceiver coupled to at least one antenna; at least one pulsed electromagnetic field (PEMF) induction coil; at least one cold laser emitter; at least one red light, infrared light, and/or heat (RIRH) emitter; and processing circuitry communicatively coupled to the at least one memory, the at least one wireless transceiver, the at least one PEMF induction coil, the at least one cold laser emitter, and the at least one RIRH emitter, wherein the processing circuitry is configured to provide a treatment to a treatment recipient, the treatment comprising at least one of PEMF therapy, cold laser therapy, infrared light therapy, red light therapy, heat therapy, or a combination thereof, sequentially, simultaneously, or a combination thereof.

In an aspect, a non-transitory computer-readable medium storing computer-executable instructions, the computer-executable instructions comprising at least one instruction for causing at least one processor to: determine, based on information associated with a treatment recipient, that a flare-up of pain and/or inflammation is in progress or that a likelihood of a flare-up of pain and/or inflammation exceeds a threshold level; and provide, to the treatment recipient, treatment to avoid or mitigate the flare-up of pain and/or inflammation, wherein the treatment comprises at least one of TENS therapy, PEMF therapy, cold laser therapy, infrared therapy, red light therapy, heat therapy, or a combination thereof, sequentially, simultaneously, or a combination thereof.

Other objects and advantages associated with the aspects disclosed herein will be apparent to those skilled in the art based on the accompanying drawings and detailed description.

### BRIEF DESCRIPTION OF THE DRAWINGS

The accompanying drawings are presented to aid in the description of various aspects of the disclosure and are provided solely for illustration of the aspects and not limitation thereof.

FIG. 1 is a block diagram illustrating an example system for treating pain and/or inflammation, according to aspects of the disclosure.

FIG. 2. is an exploded view of an example apparatus for treating pain and/or inflammation, according to aspects of the disclosure.

FIGS. 3A-3H depict embodiments of a user interface within an example software application for treating pain and/or inflammation, according to aspects of the disclosure.

FIG. 4 is a flowchart illustrating an example method for treating pain and/or inflammation, according to aspects of the disclosure.

FIG. 5 is a flowchart of an example process associated with systems and methods for treating pain and/or inflammation, according to aspects of the disclosure.

FIG. 6 illustrates entries in an example database of data that may be collected and used for flare-up detection and prediction, according to aspects of the disclosure.

### DETAILED DESCRIPTION

Disclosed are techniques for treating pain and/or inflammation. In an aspect, a method of treating pain and/or inflammation includes determining, based on information associated with a treatment recipient, that a flare-up of pain and/or inflammation (“flare-up”) is in progress or that a likelihood of a flare-up exceeds a threshold level, and providing, to the treatment recipient, treatment to avoid or mitigate the flare-up. In some aspects, the treatment recipient can select a preferred treatment. In some aspects, the treatment is provided to the treatment recipient via a portable, wearable unit that includes hardware for providing multiple therapies, such heat, cold, infrared light, transcutaneous electrical nerve stimulation, and low frequency pulsed electromagnetic field, singly, in sequence, and/or in combination. In some aspects, a software application detects the flare-up or potential flare-up via a machine learning model and provides treatment via the portable, wearable unit.

Aspects of the disclosure are provided in the following description and related drawings directed to various examples provided for illustration purposes. Alternate aspects may be devised without departing from the scope of the disclosure. Additionally, well-known elements of the disclosure will not be described in detail or will be omitted so as not to obscure the relevant details of the disclosure.

The words “exemplary” and/or “example” are used herein to mean “serving as an example, instance, or illustration.” Any aspect described herein as “exemplary” and/or “example” is not necessarily to be construed as preferred or advantageous over other aspects. Likewise, the term “aspects of the disclosure” does not require that all aspects of the disclosure include the discussed feature, advantage or mode of operation.

The systems and methods described herein are aimed at treating chronic pain and/or inflammation associated with arthritis and other ailments. The systems and methods described here include a number of treatment options, including red light therapy, pulsed electromagnetic field (“PEMF”) therapy, transcutaneous electrical nerve stimulation (“TENS”) therapy, and hot/cold temperature therapy.

Infrared (IR) light and red light can improve the circulation of oxygen-laden blood in the human body, thereby alleviating pain and promoting the faster healing of deep tissues—which is especially important for those who suffer from Psoriasis and Psoriatic arthritis (“PsA”).

TENS at low frequency can help with the inflammation, as well pain management of those with chronic arthritis. High frequency TENS is not effective for those with PsA as the electric current can damage the skin tissue and inflict more pain. The device described here combines low frequencies of TENS and PEMF to aide in the optimal pain and/or inflammation relief.

Low frequency pulsed electromagnetic field (PEMF) can provide noninvasive, safe, and easy to apply methods to treat pain, inflammation and dysfunctions associated with rheumatoid arthritis (RA) and osteoarthritis (OA). PEMF also has a long-term record of safety. PEMF can not only alleviate the pain in the arthritis condition, but it can also afford chondroprotection, exert anti-inflammatory action and help in bone remodeling.

Hot/cold therapies can also help alleviate the symptoms of PsA as heat helps increase the blood flow to loosen stiffed joints and cold decreases blood flow to help reduce inflammation. By activating heat therapy utilizing pulsation and electricity, the device described here can reduce stiffness and joint pain. Conversely discrete cold lasers can reduce puffiness and inflammation. Cold laser therapy uses low levels of energy or photons that penetrate deep into a painful tissue. The light leads to chemical changes that help damaged cells recover and regrow. Cold laser therapy has anti-inflammatory, anti-edema, and analgesic effects.

The benefits of combining all four technologies, especially TENS and Infrared light, can lead to unexpected results. TENS can relieve the muscles while both waves penetrate the body's cells to act on the metabolic system. Targeting the cells with near-infrared light can cause nitric oxide to dissociate and increase the cell's metabolism. Increasing metabolism increases the rate of healing in the region, reduces inflammation faster, and provides effective pain relief. Applying the infrared red light on top of the TENS can speed up the effects to provide relief in an expediated time frame.

The following figures depict embodiments of an example device described herein, including components of the device. The relative size and scale of components of the device, as well as the device itself, are not intended to be drawn to scale and are illustrative only. Other embodiments of the systems and methods described here will be apparent to those skilled in the art from consideration of this specification, the accompanying drawings, and practice of the systems and methods disclosed herein. It is intended that the specification and examples be considered as illustrative only. Though some of the described methods have been presented as a series of steps, it should be appreciated that one or more steps can occur simultaneously, in an overlapping fashion, or in a different order. The order of steps presented is only illustrative of the possibilities and those steps can be executed or performed in any suitable fashion. Moreover, the various features of the examples described here are not mutually exclusive. Rather any feature of any example described here can be incorporated into any other suitable example. It is intended that the specification and examples be considered as exemplary only.

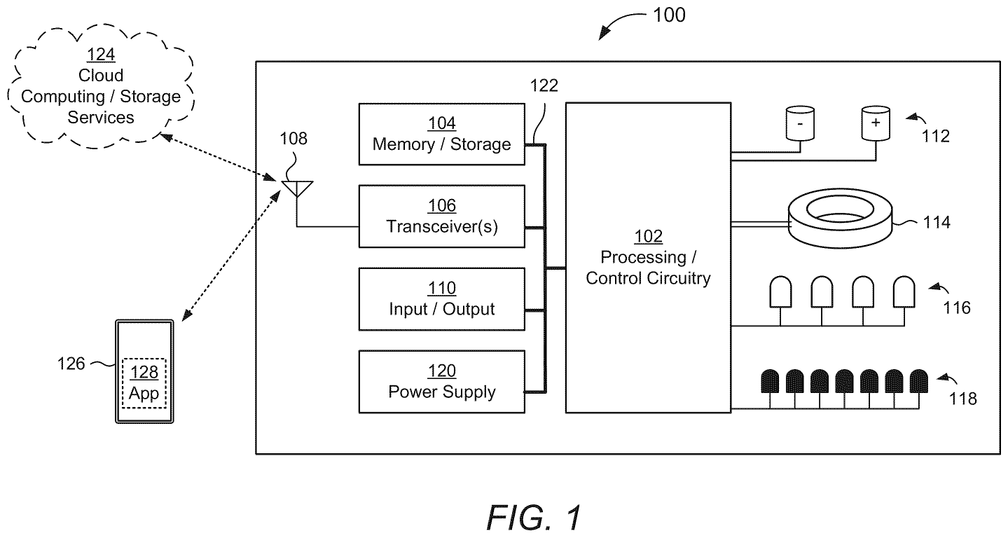

FIG. 1 is a block diagram illustrating an example system 100 for treating pain and/or inflammation, according to aspects of the disclosure.

In the example illustrated in FIG. 1, the system 100 includes processing and/or control circuitry 102, which may also be referred to herein as “controller 102”. Examples of processing and control circuitry include, but are not limited to, general purpose processors, application specific integrated circuits (ASICs), field-programmable gate arrays (FPGAs), system on chip (SoC) devices, discrete circuits, or other processing and/or control means. The controller 102 may comprise one or more integrated circuit devices and its functions may be distributed across one or more discrete chips, chiplets, dies, SoC, etc.

In the example illustrated in FIG. 1, the system 100 includes at least one memory and/or storage (memory/storage) unit 104, which may include, but are not limited to, random access memory (RAM), read-only memory (ROM), flash memory, solid-state drives (SSDs), hard disk drives (HDDs), and/or other storage means.

In the example illustrated in FIG. 1, the system 100 includes at least one transceiver 106 and at least one antenna 108 for communicating wirelessly with other devices, and by which the system 100 may communicate with an application hosted by a mobile phone, personal computer, or other computing platform separate from the system 100. Example transceivers include, but are not limited to, Wi-Fi transceivers, Bluetooth transceivers, cellular transceivers, IR transceivers, and/or other wireless communications means. In some aspects, the system 100 may use the transceiver 106 and antenna 108 to receive health information from a smart watch or other health monitoring device worn by the user.

In the example illustrated in FIG. 1, the system 100 includes at least one input/output circuit (I/O) 110. Example I/O 110 may include, but are not limited to, serial ports, parallel ports, physical or virtual keyboards, microphones, speakers, graphic user interfaces (GUIs), indicator lights, graphic displays, and/or other I/O means.

In the example illustrated in FIG. 1, the system 100 includes two or more TENS electrodes, which may be controlled individually and/or as a group. In the example shown in FIG. 1, the controller 102 controls a pair of TENS electrodes 112, labeled positive (+) and negative (−). In other aspects, the controller 102 may configure a separate TENS controller (not shown) that controls the TENS electrodes 112.

Low-frequency TENS settings typically range from 2 Hz (hertz) to 10 Hz. This setting is often used for acute or chronic pain management, muscle relaxation, and endorphin release. Low-frequency TENS is thought to stimulate the release of endorphins, the body's natural painkillers, and provide relief from persistent pain. Medium-frequency TENS settings typically range from 10 Hz to 50 Hz. Medium-frequency TENS is often used for muscle stimulation, improving circulation, and muscle re-education. It can help reduce muscle atrophy and enhance muscle strength, making it useful for physical therapy and rehabilitation. High-frequency TENS settings typically range from 50 Hz to 250 Hz or higher. High-frequency TENS is primarily used for acute pain relief and may help to reduce the perception of pain. It's often used for short-term pain management, such as during labor or after surgery. The choice of frequency setting depends on the specific goals of the TENS therapy and the type of pain or condition being treated. In some aspects, the system 100 controls the frequency and intensity (amplitude) to customize the treatment to the treatment recipient's individual needs. For example, the controller 102 may cause the TENS electrodes 112 to provide low frequency TENS at medium intensity, etc.

In the example illustrated in FIG. 1, the system 100 includes one or more PEMF induction coils, which may be controlled individually and/or as a group. In the example shown in FIG. 1, the controller 102 controls one PEMF induction coil 114. In other aspects, the controller 102 may configure a separate PEMF controller (not shown) that controls the PEMF induction coil 114.

Low-frequency PEMF settings typically range from 1 Hz (hertz) to 100 Hz or even lower. LF-PEMF is often used for promoting relaxation, sleep improvement, and addressing chronic conditions. It may have a calming effect on the body and nervous system and is believed to support overall well-being. Medium-frequency PEMF settings generally range from 100 Hz to around 1,000 Hz. MF-PEMF is commonly used for tissue regeneration, pain management, and supporting the body's natural healing processes. It may help with reducing inflammation, improving circulation, and accelerating recovery from injuries. High-frequency PEMF settings typically exceed 1,000 Hz and can go much higher. HF-PEMF is often used for acute pain management, enhancing circulation, and stimulating cellular activity. It may promote faster healing and tissue repair. The choice of frequency setting for a PEMF machine depends on the specific therapeutic goals and the type of condition being treated. In some aspects, the system 100 controls the frequency and intensity to customize the treatment to the treatment recipient's individual needs. For example, the controller 102 may cause the PEMF induction coil 114 to provide low frequency PEMF at low intensity, etc.

In the example illustrated in FIG. 1, the system 100 includes one or more cold laser emitters, which may be controlled individually and/or as a group. In the example shown in FIG. 1, the controller 102 controls four cold laser emitters 116. In other aspects, the controller 102 may configure a separate cold laser controller (not shown) that controls the cold laser emitters 116. In some aspects, the cold laser emitters 116 emit light at a wavelength of 808 nm at 500 mW of power.

In the example illustrated in FIG. 1, the system 100 includes one or more red light/IR light/heat (RIRH) emitters, which may be controlled individually and/or as a group. In the example shown in FIG. 1, the controller 102 controls seven RIRH emitters 118. In other aspects, the controller 102 may configure a separate RIRH controller (not shown) that controls the RIRH emitters 118.

In some aspects, the RIRH emitters 118 may comprise some combination of light emitting diodes (LEDs) that emit light with wavelengths of 632 nm (red), 660 nm (deep red), 850 nm (short infrared), and/or 940 nm (long infrared). Deep red light in the 660 nm range is often used in photobiomodulation therapy for various purposes, including skin rejuvenation, pain management, and wound healing. Infrared light in the 850 nm and 940 nm range can provide heat, with 940 nm IR providing better penetration through certain materials and increased range due to lower absorption by water vapor. The longer wavelength penetrates a deeper layer of tissue, for example, to enhance skin cell and muscle recovery. In some aspects, some or all of the RIRH emitters 118 may be used in a higher power mode to provide heat therapy with or instead of IR/red light therapy.

In the example illustrated in FIG. 1, the system 100 includes a power supply 120, which may comprise, but is not limited to, a battery or battery pack, a power adapter interface, a power adapter, a solar cell, and/or other power supply means.

In the example illustrated in FIG. 1, the controller 102, memory and/or storage 104, transceiver(s) 106, I/O 110, and power supply 120 are electrically and/or communicatively coupled together via wiring 122, which may include, but is not limited to, a data bus, an address bus, a serial link, a parallel link, a switching fabric, printed circuit board (PCB) traces, and/or other electrical connection means.

In some aspects, processing tasks may be performed by the controller 102 alone or in cooperation with cloud-based processing and/or storage resources 124, which may also be referred to herein as the cloud processors 124, the cloud storage 124, or simply the cloud 124.

In some aspects, the system 100 may be monitored and/or controlled by a remote device 126. Example remote devices include, but are not limited to, mobile phones, personal computers, smart watches, or other monitoring and/or controlling means. In some aspects, the system 100 may be monitored and/or controlled by a software application 128 executing on the remote device 126. The software application 128 may also be referred to herein as the software 128, the software application 128, or simply the app 128.

In some aspects, the controller 102 may execute programs and/or algorithms for providing one or more therapies to the user of the system 100. These therapies may include but are not limited to: providing TENS stimulation to the user; providing PEMF stimulation to the user; providing cold therapy to the user; providing red light therapy to the user, providing IR therapy to the user; providing heat therapy to the user; or combinations of the above simultaneously or in sequence. For example, a therapy session may include providing heat therapy, followed by providing PEMF and IF therapy simultaneously, followed by providing alternating TENS therapy and cold laser therapy, and concluding with a second round of heat therapy. The sequence of single therapies and combinations of therapies described above is illustrative and not limiting.

In some aspects, specific therapies may be pre-configured on the system 100 and selectable by the user. In some aspects, the user may create custom therapies, which are then stored locally in the memory/storage unit 104, in the cloud 124, or in the remote device 126.

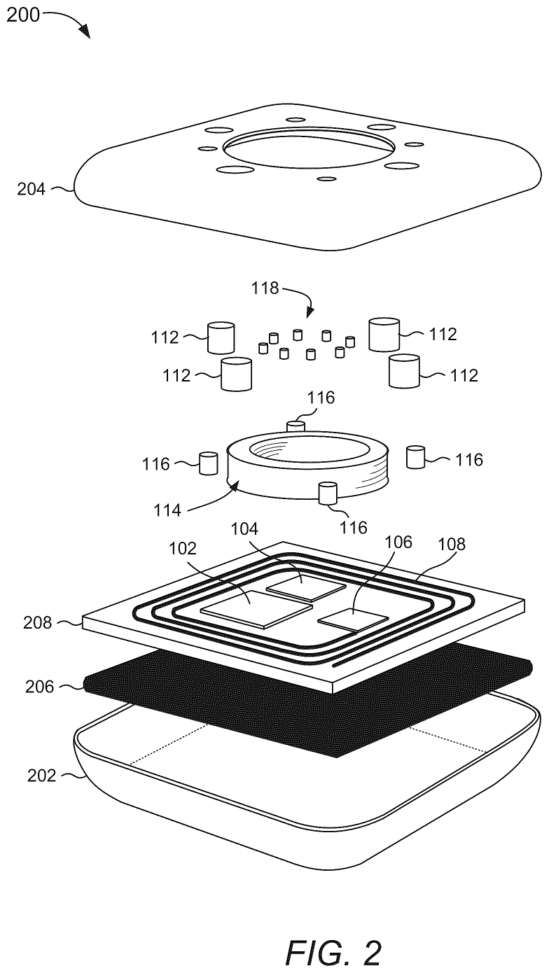

FIG. 2 is an exploded view of an example apparatus 200 for treating pain and/or inflammation, according to aspects of the disclosure. In the example illustrated in FIG. 2, the apparatus 200 includes an enclosure comprising a bottom portion 202 and a top portion 204, both of which enclose at least one battery 206 and at least one circuit board (PCB) 208. The top portion 204 has openings through which a PEMF induction coil 114 and TENS electrodes 112 protrude. Other openings provides windows through which the outputs of the cold laser emitters 116 and the RIRH emitters 118 may shine on the tissues to be treated.

Other variations of the apparatus 200 are also contemplated by the instant disclosure. For example, in some aspects, the apparatus 200 may omit the TENS electrodes 112; in some aspects, the apparatus 200 may omit the PEMF induction coil 114; and so on. Variations of the apparatus 200 may omit one or more of any of the TENS electrodes 112, the PEMF induction coil 114, the cold laser emitters 116, and RIRH emitters 118.

In some aspects, processors, memory, transceivers, antenna, etc., from FIG. 1 may be mounted to or within the PCB 208. In the example shown in FIG. 2, the controller 102, the memory/storage unit 104, the transceiver 106, and the antenna 108 are mounted to the PCB 208.

It will also be understood that the apparatus 200 shown in FIG. 2 is illustrative and not limiting, i.e., the apparatus 200 may also include additional devices, processors, emitters, or other features now shown in FIG. 2, including, but not limited to, buttons, switches, displays, lights, speakers, microphones, power supply or other jacks, interfaces, haptic devices, inductive charging coils, and other input, output, or I/O devices. For example, in some aspects, the apparatus 200 may indicator lamps or a display to indicate status of operation, battery charge, or other indicia of operation.

In some aspects, the apparatus 200 may be attached to medical grade electrode pads, e.g., via a jack or other electrical interface. In some aspects, the medical grade electrode pads may be coated in a topical analgesic for additional pain relief. In some aspects, the electrode pads may be available in different shades of flesh tones so that the pads are less noticeable/more discrete.

In some aspects, the relatively small and compact size of the apparatus 200 allows it to be secured to the user by medical tape or other medical adhesive means. In some aspects, the apparatus 200 may be secured to the user by a strap, sleeve, wrap, or other non-adhesive means.

The apparatus 200 provides a number of advantages over the prior art. For example, in some aspects, the apparatus 200 is a noninvasive wearable device for treating chronic pain and/or inflammation that is affordable, not bulky, and does not require numerous devices. The apparatus 200 can administer multiple therapies to address both arthritis symptoms and treatment side effects. Implementing all four technologies in a wearable medical therapy device such as apparatus 200 can provide someone suffering from chronic arthritis with control and better results tailored to their unique needs. The integrated technologies and discrete wearable functionality of the device can allow a user to perform day-to-day activities with minimal disturbances. For example, an individual may have a flare-up where she experiences pain and stiffness in a wrist. Pain medicine and other remedies will not be fast enough to provide relief or relief will come at the cost of being unable to perform day-to-day functions, and commonly prescribed pain medications can reduce thought clarity and can cause side effects like dizziness and nausea.

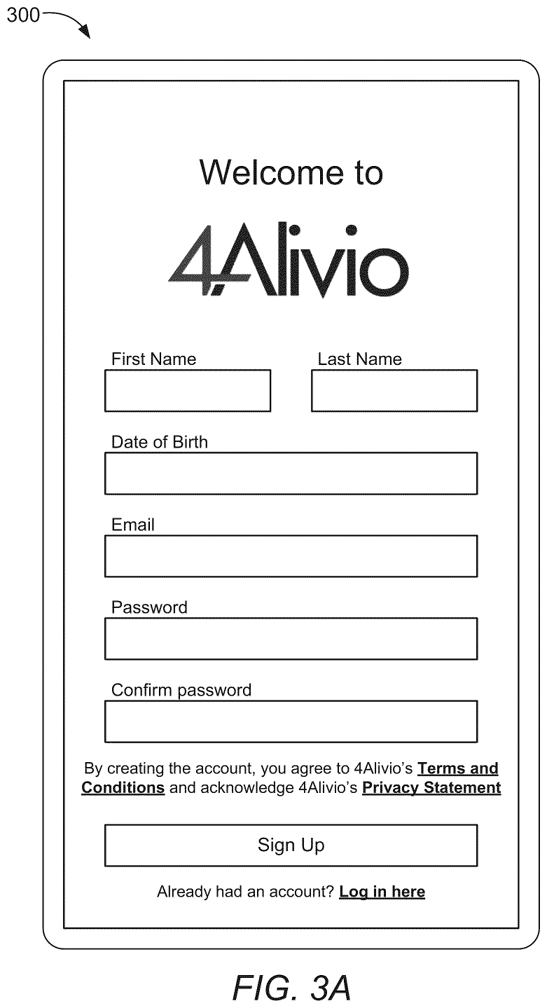

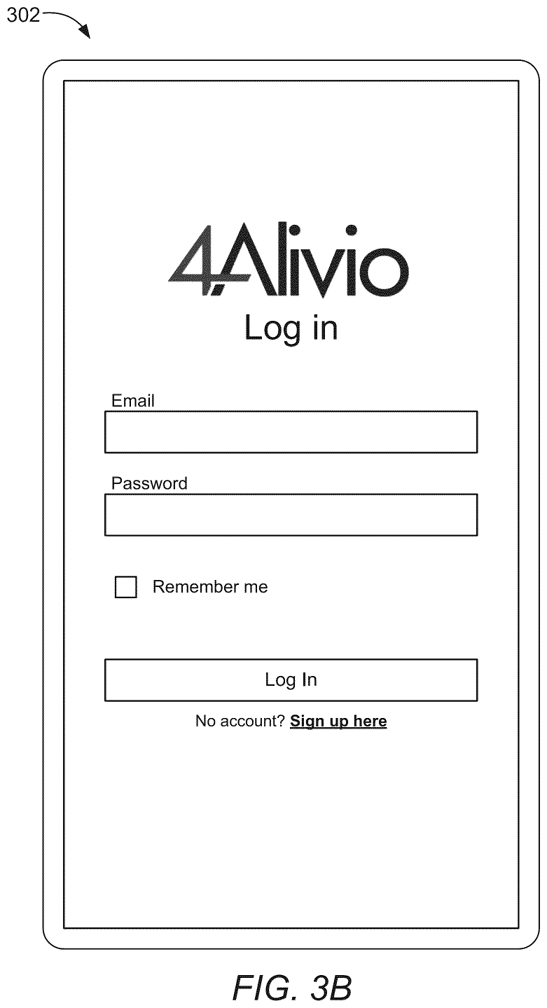

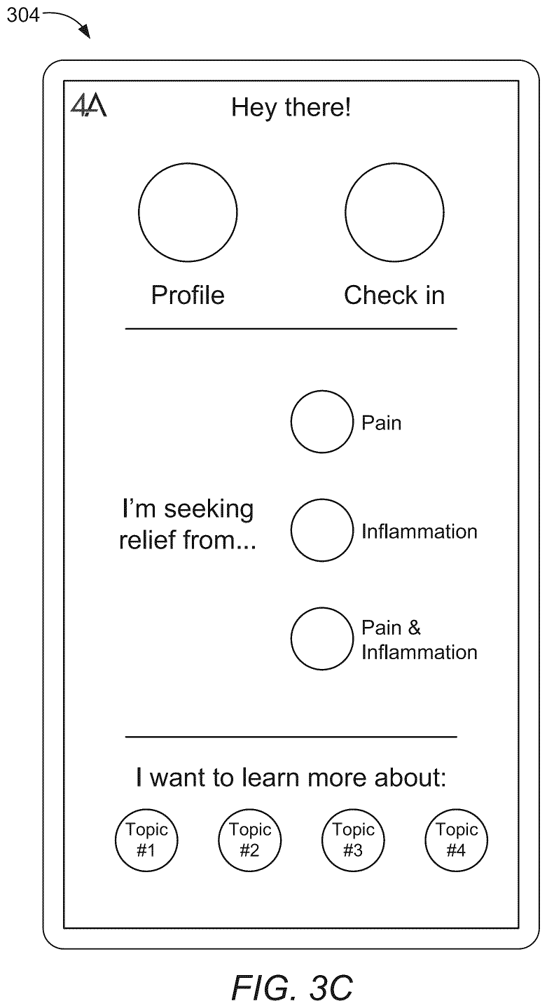

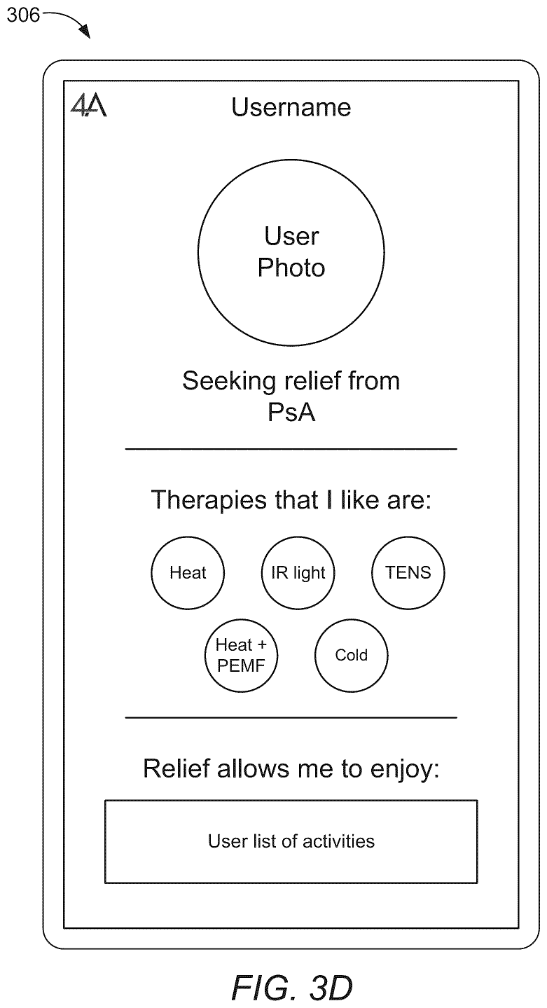

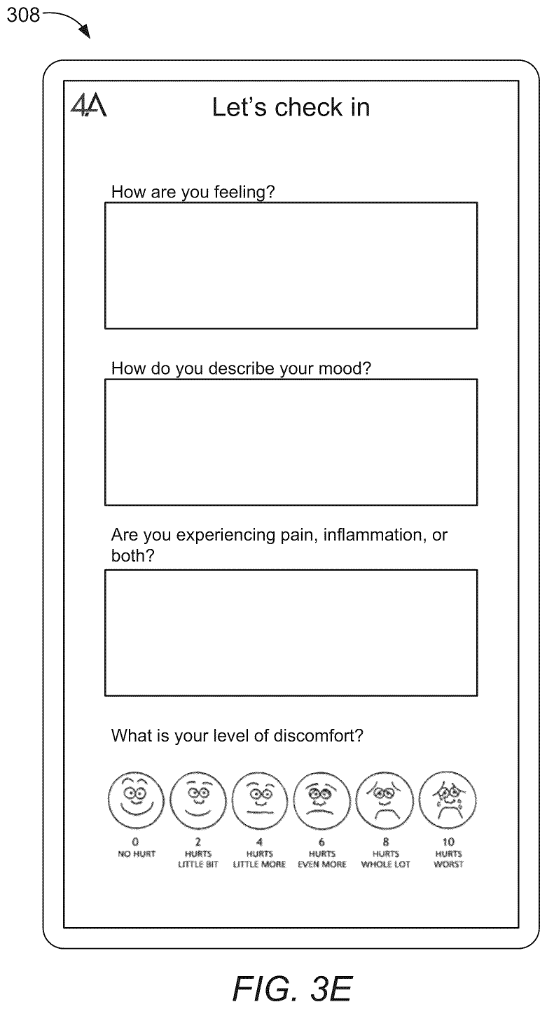

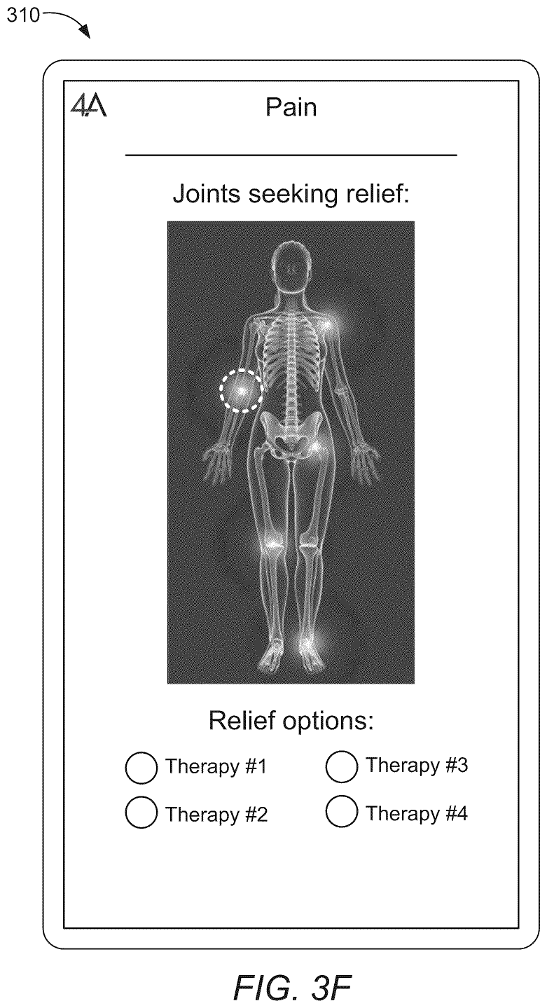

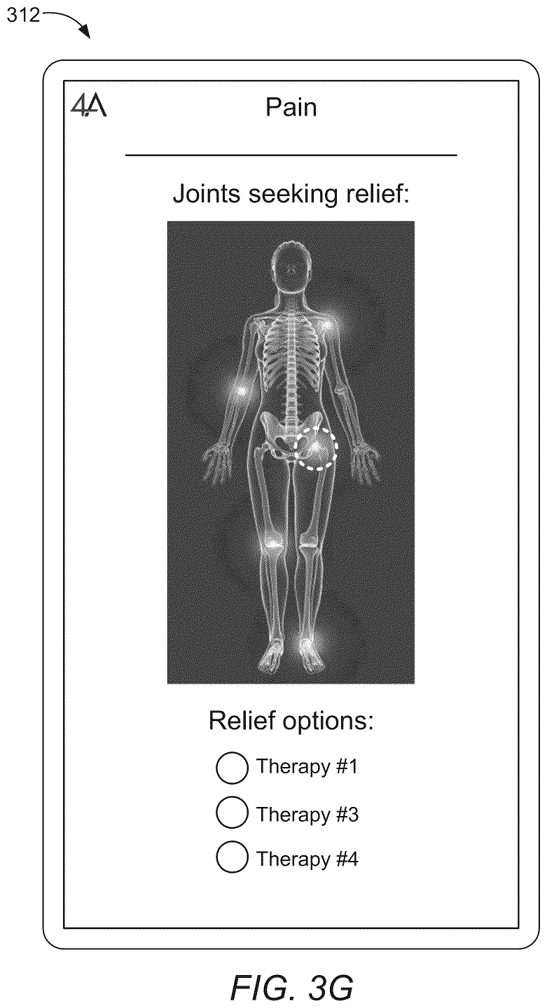

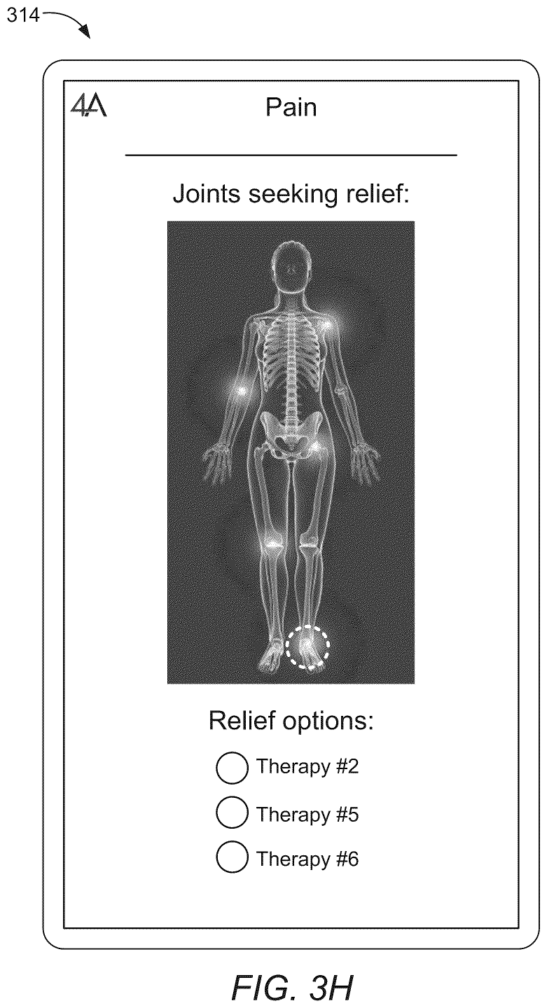

FIGS. 3A-3H depict embodiments of a user interface (“UI”) depicted within an example software application for treating pain and/or inflammation (e.g., software application 128), according to aspects of the disclosure. In some aspects, the software application 128 may be executed on a remote device 126. From these screens, a user can select a therapy options (e.g., IR light, TENS, PEMF, hot/cold), therapy locations or sites (e.g., elbow joint or knee joint), and one or more therapy routines to be delivered (i.e., an order or combination of the therapy options). The user will also be able to initiate the therapy session from the application. In some embodiments, after a therapy session, a user can provide feedback regarding the effectiveness of the session. The software application can save this feedback information for later use in suggesting future therapy routines or protocols to the user and/or other users.

FIG. 3A is a depiction of an example entry page 300, with which the user can sign up for an account or log into an existing account. This page may be displayed if the user does not have an account or is logging in for the first time using a new device.

FIG. 3B is a depiction of an example log in page 302. This page may be displayed if the user already has an account and is logging in from a previously used device.

FIG. 3C is a depiction of an example main page 304. In some aspects, this page has links to a user profile page, a check in page, and a therapy page, all of which are described in more detail below. In some aspects, this page has links to pages devoted to other topics that the user may find to be of interest.

FIG. 3D is a depiction of an example user profile page 306. In some aspects, this page may display a user profile picture or other user profile information. In some aspects, this page may display a statement of purpose, e.g., “Seeking relief from PsA”. In some aspects, this page may display one or more therapies that are available to the user, or a subset of those therapies that the user has already selected as desired therapies. In some aspects, this page may display a subset of available therapies that are recommended for sufferers of the particular ailment identified by the user (e.g., PsA). In some aspects, this page may display desired goals or intended benefits of therapy, e.g., “Relief allows me to enjoy jogging,” or other motivational materials.

FIG. 3E is a depiction of an example check in page 308. In some aspects, this page is a means for the user to record information about the user's physical, mental, and/or emotional state. For example, in some aspects, this page provides the user a way to indicate how the user is feeling, what the user's mood is, whether the user is experiencing pain, inflammation, or both, and allows the user to indicate the user's current level of discomfort. Examples of information that the user may supply via the check in page 308 include, but are not limited to, pain information (e.g., presence or absence, location, type, severity), inflammation information (e.g., presence or absence, location, type, severity), and an indication of the user's tiredness or level of fatigue.

### Data Collection/Analysis

In some aspects, the information provided by the check in page 308 may be used to collect user data that is used to create a historical record of pain and/or inflammation. In some aspects, in addition to the information provided by the user via the check in page 308, other information may become part of the historical record, such as other information about the user and information about the environment.

Examples of other information about the user include, but are not limited to: vital signs and other biometric information, such as heart rate, body temperature, oxygen saturation levels, glucose level, etc., which may be received from a smart watch or health monitor device worn by the user; whether the user is currently moving or stationary; the time of the user's last meal; nutrition information, such as caloric intake, saturated fat, high glycemic intake, high fiber, protein, Omega-3 fatty acids, calcium, magnesium, resveratrol, coenzyme Q3, turmeric, garlic, ginger intake, as well as vitamin D and multivitamin/antioxidant levels, which may be directly provided by the user or calculated from a list of foods that the user indicates has been eaten by the user.

In some aspects, other information about the user may include but are not limited to: current and planned medication regimens, including therapeutic infusions; scheduled medical appointments and future visits; herbal and alternative medicine, including planned approaches. In some aspects, other information about the user may include indicia of healing and functionality, which may be guided by inflammation markers, such as C-reactive protein (CSR), erythrocyte sedimentation rate (ESR), white blood cell count, cortisol levels, and other indicia. In some aspects, other information about the user may include indicia of range of motion, which may be indicated by physical therapy exercises, such as knee and back flexion/extension, knee and back rotational movements, arm/forearm flexion/extension, neck/spine flexion/extension, and other indicia. In some aspects, other information about the user may include indicia of disease progression, which may be evaluated through the physical appearance of cutaneous psoriasis, for example.

Examples of information about the environment include but are not limited to: current date and time, local temperature (AM, PM, high, low, average, delta), wind chill factor, heat index, local humidity (AM, PM, high, low, average, percentage change), barometric pressure, and chance of precipitation, some or all of which may be harvested from a weather forecast website, for example.

### Detection and Prediction

In some aspects, this historical record may be analyzed to identify trends, e.g., the user tends to have more pain in the morning than in the evening, the user tends to have more inflammation on cold days rather than hot days, etc. In some aspects, a logic regression or machine learning (ML) model or other analysis technique may be used to detect and/or predict flare-ups. In some aspects, the model may be instantiated as part of the software application. In some aspects, the model may reside on a cloud processing server with which the software application communicates via the internet. In some aspects, the model may be an ML model that has been trained using data from a particular user. In some aspects, anonymized information aggregated from multiple users may alternatively or also be considered during analysis. For example, in some aspects, aggregate data from multiple users suffering different ailments may be part of a training set for an ML model.

In some aspects, the controller 102 may execute programs and/or algorithms for detecting indications that the user is starting to have a flare-up, e.g., so that the user may be notified of potential treatment options to alleviate symptoms or mitigate the severity of the symptoms. For example, in some aspects, the software application may receive biometric data from a smart watch being worn by the user, and determine, based on a variety of factors (e.g., time of day, current temperature, the user's most recent check in status, the user's historical trends, etc.) that the user is starting to show symptoms of a flare-up.

In some aspects, the controller 102 may execute programs and/or algorithms for predicting or anticipating that the user is likely to have a flare-up, e.g., so that the user may take proactive steps to prevent or minimize the symptoms of the predicted flare-up. Example algorithms include, but are not limited to:
Random Forest: This ensemble learning method offers high accuracy and can deal with categorical and numerical features. It's robust to overfitting and can capture complex relationships in the data, making it a strong candidate for this task.
Support Vector Machines (SVM): With the right kernel, SVM can model nonlinear relationships between the weather and biometric factors. It's especially useful when there's a clear margin of separation in the data.
Neural Networks: Deep Learning methods, particularly Recurrent Neural Networks (RNN) or Long Short-Term Memory (LSTM) networks, could model sequences in the data, such as weather patterns over 0 me, combined with the patient's historical data, to predict flare-ups.

The algorithms mentioned above learn from data to accurately predict conditions. One feature of these algorithms is that they may maintain and use past relationships to predict future conditions, which is one of the strengths of these algorithms for a successful flare-up prediction. For example, RNN has a memory from the past that relates to the present to predict what is coming next. In some aspects, the method selection would be based on exploratory data analysis, understanding the relationships between variables, and cross-validation performance. In some aspects, the above methods are combined to create an ensemble model, leveraging the strengths of different algorithms to achieve a more robust and accurate prediction.

An example of a flare-up prediction algorithm is illustrated in Table 1, below:

TABLE 1

| | Humidity | | | | | |
|---|---|---|---|---|---|---|
| | Daily Change | | Rating | | | Flare Up |
| Day | AM | PM | AM | PM | Sum | Prediction |
| 1 | | | | | 0 | No |
| 2 | | 8% | 0 | 0 | 0 | No |
| 3 | −12% | −7% | 1 | 0 | 1 | No |
| 4 | −5% | 2% | 0 | 0 | 0 | No |
| 5 | −19% | −31% | 1 | 1 | 2 | Yes |
| 6 | 2% | 2% | 0 | 0 | 0 | No |
| 7 | −2% | −2% | 0 | 0 | 0 | No |
| 8 | 11% | 29% | 1 | 1 | 2 | Yes |

In this non-limiting example, it has been determined that for a particular user (and possibly for other users as well), there is a correlation between humidity and likelihood of a flare-up. Specifically, it has been determined that flare-ups are related to significant changes in humidity over the course of a full day. In some aspects, this correlation may have been identified as a result of analysis of historical data from the user and/or other users. In some aspects, this correlation may have been identified via machine learning (ML) techniques, such as decision trees and logistic regression.

Thus, in this example, a morning (AM) or evening (PM) humidity rating is calculated as having a value of 1 if the humidity rises or falls more than 10% over the course of that half-day. For example, on day 3 in Table 1, above, the humidity fell by more than 10% during the morning but fell by less than 10% during the evening, so the AM humidity rating is 1 and the PM humidity rating is 0. The sum of the AM and PM humidity ratings for day 3 is not more than 1, so a flare-up is not predicted. On day 8, however, both the AM humidity and PM humidity increased by more than 10%, so the sum of the AM and PM humidity ratings for day 8 is more than 1, so a flare-up is predicted. In some aspects, the flare-up prediction may produce a more granular output, e.g., not only predicting a flare-up, but also predicting where the flare-up will occur, how severe it is predicted to be, or other indications.

It will be understood that his example is illustrative and not limiting, and that other algorithms may be employed to predict flare-ups. For example, an ML approach may identify a correlation between the flare-up prediction result and other information provided by the user, such as a pain rating, user tiredness, etc., to enhance prediction accuracy. In some aspects, for example, a neural network may accept as input a wide variety of factors and may be trained to accurately predict a flare-up even without a clear human understanding of the drivers and causes of a flare-up. In such cases it is still possible to derive potential mitigation actions that might be taken to avoid a predicted flare-up based on analysis of the training set, personal insights by the user, and other means. For example, the user may be provided with suggestions to help avoid or mitigate flare-ups, e.g., “It's cold outside—try to stay warm!” In some aspects, the user may be prompted to seek treatment. In some aspects, prompting the user to seek treatment may include presenting the user with a treatment page or prompting the user to navigate to the treatment page within the software application.

In some aspects, the detection algorithms and/or models may be used to detect indicia of inflammation markers, loss of flexibility, and/or disease progression, and may optionally notify the treatment recipient of these indicia (e.g., “Your blood pressure seems to be increasing”) or what those indicia may mean (e.g., “You seem to be losing range of motion in your neck/back/arms/shoulders/knees/etc.”).

### Treatment

FIG. 3F is a depiction of an example treatment page 310. In some aspects, this page allows the user to identify a location of pain, inflammation, or discomfort, and initiate a treatment using a therapeutic device, such as the apparatus 200. In the example shown in FIG. 3F, the user has selected the user's right elbow as the source of pain and/or inflammation. In response, the software application displays a set of one or more available therapies. In the example shown in FIG. 3F, three therapies are presented, labeled “Therapy #1”, “Therapy #2”, “Therapy #3”, and “Therapy #4”, but other numbers of therapies may be presented instead, depending on the user's preferences, history, or other factors.

For illustrative purposes only, here is an example of four different therapy options that might be displayed on example treatment page 310. In this example, the therapy options may be presented as therapy for inflammation, therapy for pain, therapy for inflammation and pain, and a custom therapy option, with the details of each therapy being as shown in Table 2, below:

TABLE 2

| | |
|---|---|
| Therapy #1 | Inflammation and Pain Relief |
| | Heat (1 minute) |
| | IR light (4 minutes) |
| | Alternating TENS and PEMF (10 minutes) |
| | Cold (5 minutes) |
| Therapy #2 | Pain Relief |
| | Heat (1 minute) |
| | IR light (4 minutes) |
| | Cold (5 minutes) |
| Therapy #3 | Inflammation Relief |
| | Cold laser |
| | Soothing heat |
| | TENS, low frequency |
| | Constant infrared light |
| Therapy #4 | Pain Relief |
| | Pulsating infrared light |
| | TENS, low-mid frequency, longer duration |
| | PEMF, low frequency |
| | TENS & PEMF alternate while the infrared light remains constant |
| Therapy #5 | Inflammation and Pain Relief |
| | TENS |
| | PEMF |
| | Infrared red light |
| | Cold Laser |
| | Soothing heat |
| | 4 technology circuit of treatment beginning with infrared |
| | (constant to pulse to constant), TENS, PEMF, and hot/cold |
| | alteration between TENS & PEMF |
| Therapy #6 | Custom Therapy |
| | User-selected cold/hot/IR/TENS/PEMF/etc. |
| | User-selected sequence, combination, duration, etc. |

For illustrative purposes only, here is another example of four different therapy options that might be displayed on example treatment page 310. In some aspects, the therapy options may be presented as therapy using heat, therapy using cold, therapy using PEMF, therapy using TNES, or therapy using combinations of the above, as shown in Table 3, below:

TABLE 3

| | |
|---|---|
| Therapy #1 | Heat |
| | Constant heat, with preset warm-up and cool-down periods |
| Therapy #2 | Cold |
| | Intermittent cold treatment, with preset period and duty cycle |
| Therapy #3 | PEMF |
| | Frequency A, then Frequency B, then off |
| Therapy #4 | TENS + IR |
| | Low intensity TENS, pulsating IR light |

In some aspects, additional treatment options may be suggested to a treatment recipient. Examples of additional treatment options include, but are not limited to: herbal treatments and other alternative medicine approaches; physical therapy exercises to improve range of motion, muscle tone, circulation, etc.; and other treatments.

FIG. 3G is a depiction of an example treatment page 312. In the example shown in FIG. 3G, the user has selected the user's left hip as the source of pain and/or inflammation, and three therapies are presented, labeled “Therapy #1”, “Therapy #3”, and “Therapy #4”. This example illustrates the point that Therapy #1 may be good for both elbow and hip, but Therapy #5 and Therapy #6 may be better for the hip than Therapy #2 or Therapy #3, i.e., that the selection of therapies may differ based on the affected area, a user-stated preference, and/or other selection criteria.

FIG. 3H is a depiction of an example treatment page 314. In the example shown in FIG. 3H, the user has selected the user's left ankle as the source of pain and/or inflammation, and three therapies are presented, labeled “Therapy #2”, “Therapy #5”, and “Therapy #6”. In the example shown in FIG. 3H, the software application presents a different subset of available therapies based on the affected area, user preferences, and/or other selection criteria.

In some aspects, a user may use multiple units simultaneously, each unit comprising an apparatus 200 able to provide multiple therapies independently. For example, a user may place each unit on a problem area (e.g., one on a wrist, one on a knee, two on the lower back, etc.), and each unit can provide the same therapies as each other or different therapies from each other. Through the application's user interface, the user can select the flare-up symptoms (e.g., inflammation, pain, stiffness, redness of skin, skin feeling hot, etc.) and the user can choose from a selection of suggested therapies or self-select treatments (e.g., any combination of one or more of IR light, PEMF, TENS, and Hot/Cold).

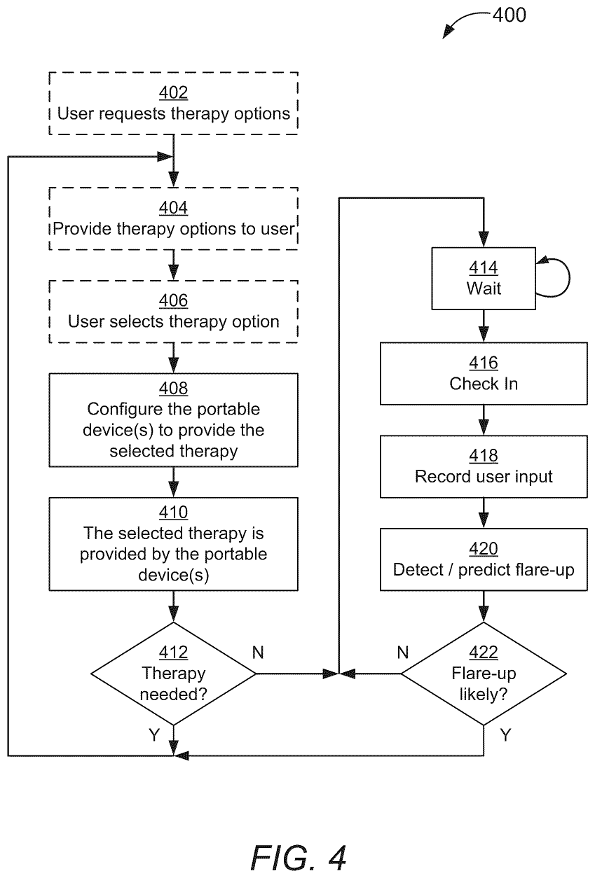

FIG. 4 is a flowchart illustrating an example method 400 for treating pain and/or inflammation, according to aspects of the disclosure. In the example shown in FIG. 4, the user requests therapy options (block 402). In some aspects, the user may do so using the treatment page 310 of the software application 128, for example, to identify the afflicted area.

In the example shown in FIG. 4, the treatment recipient may be provided with therapy options (optional block 404). In some aspects, the software application 128 may display a set of treatment options on the treatment page 310.

In the example shown in FIG. 4, the treatment recipient may select a therapy option (optional block 406). In some aspects, the treatment recipient may do so by selecting a therapy option from a list of options displayed on the treatment page 310.

It will be understood that in some scenarios, it may be desirable for treatment to be provided without interaction from the treatment recipient, such as when the user of a treatment device is sleeping, unconscious, or desires that the device provide treatment without interaction from the user. In those scenarios, optional blocks 404 and 406 may be omitted or skipped. Automatic treatment in this manner also allows this method to be used to treat animal patients.

In the example shown in FIG. 4, each of one or more portable devices is configured to provide the selected therapy or therapies (block 408). In some aspects, the software application 128 may communicate with a apparatus 200 and configure the apparatus 200 to provide the selected therapy or therapies, e.g., TENS, PEMF, cold laser, red light, IR light, heat, etc.)

In the example shown in FIG. 4, the selected therapy or therapies are provided by the portable device(s) (block 410). In some aspects, the apparatus 200 may provide the therapies using its on-board controller(s).

In the example shown in FIG. 4, the treatment recipient may then indicate that more therapies are needed (block 412), in which case the process returns to block 404. In some aspects, the software application 128 may ask the treatment recipient whether additional therapies are needed.

In the example shown in FIG. 4, if no additional therapy is needed, then the process enters a wait state (block 414), where it waits for some configured amount of time before requesting a user check in (block 416).

In the example shown in FIG. 4, the information provided by the treatment recipient during check in is recorded (block 418). In some aspects, the software application 128 may store the information locally, to a cloud server 124, or both. The information stored may also include environmental information determined from sensors on the apparatus 200 or the remote device 126, from web-based sources, or other sources of environmental information. The information stored may also include biometric information associated with the treatment recipient, e.g., from a smart watch or other health monitoring device worn by or in proximity to the treatment recipient.

In the example shown in FIG. 4, a flare-up detection and/or prediction operation may be performed (block 420). In some aspects, a flare-up may be detected or predicted based on the treatment recipient information recorded at block 418, based on environmental conditions only, based on other factors, or some combination thereof.

In the example shown in FIG. 4, it is determined whether or not a flare-up is in progress and/or is likely (block 422). In some aspects, a flare-up prediction may be made by an algorithm, a trained ML model, or other type of prediction engine running on the apparatus 200, the remote device 126, and/or the cloud processing resources 124. If a flare-up is determined to be in progress or likely, the process goes to block 404. Otherwise, the process returns to the wait state at block 414.

It will be understood that, in the example shown in FIG. 4, the treatment recipient may request therapy at any time, whether or not the method is currently in the wait state at block 414. In the event that the treatment recipient requests therapy, the process will jump to block 404 and continue from there.

It will also be understood that, in some aspects, the flare-up detection and/or prediction operation in block 420 may operate continually or periodically and may also be triggered by events other than the treatment recipient check in at block 416. For example, in some aspects, the software application 128 may continually monitor biometric data associated with the treatment recipient and may identify indications that a flare-up may be in progress. In some aspects, the software application 128 may continually monitor environmental conditions and may identify conditions that historically cause flare-ups for the particular treatment recipient. In either example above, the software application 128 may notify the treatment recipient that therapy may be needed, regardless of which step of process 400 is currently being executed.

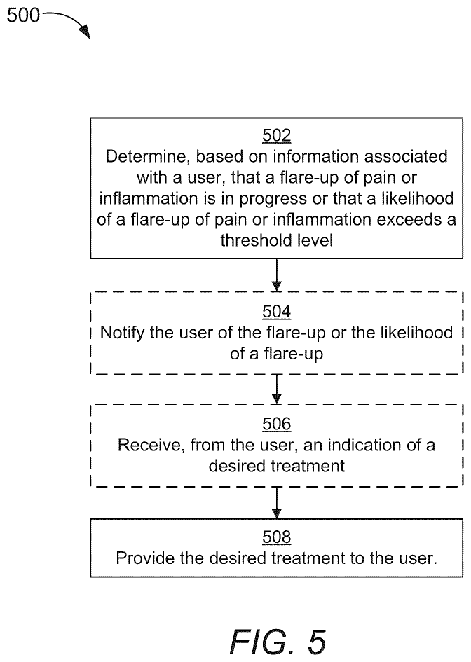

FIG. 5 is a flowchart of an example process 500 associated with systems and methods for treating pain and/or inflammation, according to aspects of the disclosure. In some implementations, one or more process blocks of FIG. 5 may be performed by one or more components of a system 100, e.g., by a software application 128 that controls an apparatus 200. Additionally, or alternatively, one or more process blocks of FIG. 5 may be performed by one or more components of the system 100 or apparatus 200, such as a processor, memory, or transceiver, any or all of which may be means for performing the operations of process 500.

As shown in FIG. 5, process 500 may include, at block 502, determining, based on information associated with a treatment recipient, that a flare-up of pain and/or inflammation is in progress or that a likelihood of a flare-up of pain and/or inflammation exceeds a threshold level. Means for performing the operation of block 502 may include the processor(s), memory, or transceiver(s) of any of the apparatuses described herein. For example, the software application 128 may determine, based on information associated with a treatment recipient, that a flare-up of pain and/or inflammation is in progress or that a likelihood of a flare-up of pain and/or inflammation exceeds a threshold level.

As further shown in FIG. 5, process 500 may optionally include, at optional block 504, notifying the treatment recipient of the flare-up or the likelihood of a flare-up. Means for performing the operation of block 504 may include the processor(s), memory, or transceiver(s) of any of the apparatuses described herein. For example, the software application 128 may notify the treatment recipient of the flare-up or the likelihood of a flare-up.

As further shown in FIG. 5, process 500 may optionally include, at optional block 506, receiving, from the treatment recipient, an indication of a selected treatment. Means for performing the operation of block 506 may include the processor(s), memory, or transceiver(s) of any of the apparatuses described herein. For example, the software application 128 may receive, from the treatment recipient, an indication of a selected treatment, using a touchscreen or keyboard of the remote device 126.

As further shown in FIG. 5, process 500 may include, at block 508, providing the selected treatment to the treatment recipient. Means for performing the operation of block 508 may include the processor(s), memory, or transceiver(s) of any of the apparatuses described herein. For example, the software may provide the selected treatment to the treatment recipient, by configuring the apparatus 200 to perform the selected treatment.

In some aspects, providing the selected treatment to the treatment recipient comprises providing TENS therapy, PEMF therapy, cold laser therapy, IR therapy, or a combination thereof, sequentially, simultaneously, or in any combination.

In some aspects, information associated with the treatment recipient comprises information about a current or previous physical, mental, and/or emotional state of the treatment recipient.

In some aspects, the information about the current or previous physical, mental, and/or emotional state of the treatment recipient comprises information indicating a level or location of pain, a level or location of inflammation, a level of hunger, a level of fatigue, a mood, or a combination thereof.

In some aspects, information associated with the treatment recipient comprises information about a current physical environment of the treatment recipient.

In some aspects, the information about the current physical environment of the treatment recipient comprises temperature, humidity, barometric pressure, precipitation, time of day, day of week, season, month, day, year, or a combination thereof.

In some aspects, determining, based on the information associated with the treatment recipient, that a flare-up of pain and/or inflammation is in progress or that a likelihood of a flare-up exceeds a threshold level comprises determining a flare-up and/or predicting a flare-up using a machine learning model that uses the information associated with the treatment recipient as an input.

In some aspects, notifying the treatment recipient of the flare-up or the likelihood of a flare-up comprises the treatment recipient via a software application.

In some aspects, receiving the indication of the selected treatment comprises receiving the indication via the software application.

In some aspects, providing the selected treatment to the treatment recipient comprises providing the selected treatment using a treatment unit.

In some aspects, providing the selected treatment using a treatment unit comprises providing the selected treatment using a treatment unit that provides TENS therapy, PEMF therapy, cold laser therapy, IR therapy, or a combination thereof, sequentially, simultaneously, or in any combination.

Process 500 may include additional implementations, such as any single implementation or any combination of implementations described below and/or in connection with one or more other processes described elsewhere herein. Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

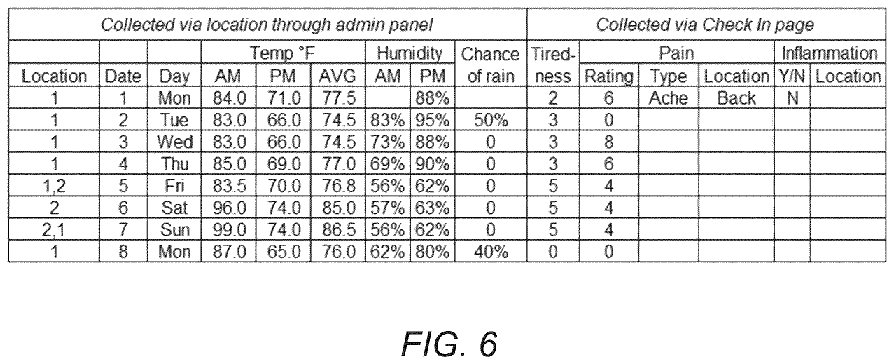

FIG. 6 illustrates entries in an example database of data that may be collected and used for flare-up detection and prediction, according to aspects of the disclosure. As shown in FIG. 6, the data may include data collected by the software application 128 with or without the treatment recipient's input (e.g., treatment recipient's location, date, time, temperature, humidity, chance of rain, etc.) and data provided by the treatment recipient (e.g., treatment recipient's tiredness, pain, and inflammation, etc.). As described herein, in some aspects, this information may be used as input into an algorithm to detect and/or predict flare-ups; in some aspects, this information may be used to train an ML model, and/or may be provided to a trained ML model to detect and/or predict flare-ups.

In the detailed description above it can be seen that different features are grouped together in examples. This manner of disclosure should not be understood as an intention that the example clauses have more features than are explicitly mentioned in each clause. Rather, the various aspects of the disclosure may include fewer than all features of an individual example clause disclosed. Therefore, the following clauses should hereby be deemed to be incorporated in the description, wherein each clause by itself can stand as a separate example. Although each dependent clause can refer in the clauses to a specific combination with one of the other clauses, the aspect(s) of that dependent clause are not limited to the specific combination. It will be appreciated that other example clauses can also include a combination of the dependent clause aspect(s) with the subject matter of any other dependent clause or independent clause or a combination of any feature with other dependent and independent clauses. The various aspects disclosed herein expressly include these combinations, unless it is explicitly expressed or can be readily inferred that a specific combination is not intended (e.g., contradictory aspects, such as defining an element as both an electrical insulator and an electrical conductor). Furthermore, it is also intended that aspects of a clause can be included in any other independent clause, even if the clause is not directly dependent on the independent clause.

Implementation examples are described in the following numbered clauses:

Clause 1. A method of treating pain and/or inflammation, the method comprising: determining, based on information associated with a treatment recipient, that a flare-up of pain and/or inflammation is in progress or that a likelihood of a flare-up of pain and/or inflammation exceeds a threshold level; and providing, to the treatment recipient, treatment to avoid or mitigate the flare-up of pain and/or inflammation.

Clause 2. The method of clause 1, wherein information associated with the treatment recipient comprises information about a current physical environment of the treatment recipient.

Clause 3. The method of clause 2, wherein the information about the current physical environment of the treatment recipient comprises temperature, humidity, barometric pressure, precipitation, time of day, day of week, season, month, day, year, or a combination thereof.

Clause 4. The method of any of clauses 1 to 3, wherein information associated with the treatment recipient comprises information about a current or previous physical, mental, and/or emotional state of the treatment recipient.

Clause 5. The method of clause 4, wherein the information about the current or previous physical, mental, and/or emotional state of the treatment recipient comprises information indicating a level or location of pain, a level or location of inflammation, a level of hunger, a level of fatigue, a mood, or a combination thereof.

Clause 6. The method of any of clauses 1 to 5, wherein determining, based on the information associated with the treatment recipient, that a flare-up of pain and/or inflammation is in progress or that a likelihood of a flare-up exceeds a threshold level comprises determining a flare-up and/or predicting a flare-up using a machine learning model that uses the information associated with the treatment recipient as an input.

Clause 7. The method of any of clauses 1 to 6, wherein providing treatment to the treatment recipient comprises providing the treatment using a treatment unit comprising circuitry for providing transcutaneous electrical nerve stimulation (TENS), pulsed electromagnetic field (PEMF), cold laser emissions, infrared (IR) light, red light emissions, and heat.

Clause 8. The method of clause 7, wherein providing treatment using the treatment unit comprises providing TENS therapy, PEMF therapy, cold laser therapy, IR therapy, red light therapy, heat therapy, or a combination thereof, sequentially, simultaneously, or a combination thereof.

Clause 9. The method of any of clauses 1 to 8, wherein providing treatment to the treatment recipient comprises providing the treatment using a treatment unit comprising circuitry for providing PEMF, cold laser emissions, IR light, red light emissions, and heat.

Clause 10. The method of clause 9, wherein providing treatment using the treatment unit comprises providing PEMF therapy, cold laser therapy, IR therapy, red light therapy, heat therapy, or a combination thereof, sequentially, simultaneously, or a combination thereof.

Clause 11. The method of any of clauses 1 to 10, wherein providing treatment to the treatment recipient comprises: notifying the treatment recipient of the flare-up or the likelihood of a flare-up; receiving, from the treatment recipient, an indication of a selected treatment to avoid or mitigate the flare-up of pain and/or inflammation; and providing the selected treatment to the treatment recipient.

Clause 12. The method of clause 11, wherein notifying the treatment recipient of the flare-up or the likelihood of a flare-up comprises notifying the treatment recipient via a software application and wherein receiving, from the treatment recipient, the indication of the selected treatment comprises receiving the indication via the software application.

Clause 13. An apparatus for treating pain and/or inflammation, the apparatus comprising: at least one memory; at least one wireless transceiver coupled to at least one antenna; at least one PEMF induction coil; at least one cold laser emitter; at least one red light, infrared light, and/or heat (RIRH) emitter; and processing circuitry communicatively coupled to the at least one memory, the at least one wireless transceiver, the at least one PEMF induction coil, the at least one cold laser emitter, and the at least one RIRH emitter, wherein the processing circuitry is configured to provide a treatment to a treatment recipient, the treatment comprising at least one of PEMF therapy, cold laser therapy, infrared light therapy, red light therapy, heat therapy, or a combination thereof, sequentially, simultaneously, or a combination thereof.

Clause 14. The apparatus of clause 13, further comprising at least one pair of TENS electrodes communicatively coupled to the processing circuitry, wherein the processing circuitry is further configured to provide TENS therapy singly or in combination with other therapies, sequentially, simultaneously, or a combination thereof.

Clause 15. The apparatus of any of clauses 13 to 14, wherein the processing circuitry is further configured to provide the treatment to the treatment recipient in response to instructions received via at least one of the wireless transceiver or an input device on or within the apparatus.

Clause 16. The apparatus of any of clauses 13 to 15, wherein the processing circuitry is further configured to provide the treatment to the treatment recipient in response to determining, based on information associated with the treatment recipient, that a flare-up of pain and/or inflammation is in progress or that a likelihood of a flare-up exceeds a threshold level.

Clause 17. The apparatus of clause 16, wherein information associated with a treatment recipient comprises at least one of: information about the treatment recipient's current or previous physical, mental, and/or emotional state; or information about a current physical environment of the treatment recipient.

Clause 18. The apparatus of any of clauses 16 to 17, wherein, to provide treatment to the treatment recipient, the processing circuitry is further configured to: notify the treatment recipient of the flare-up or the likelihood of a flare-up; receive, from the treatment recipient, an indication of a selected treatment to avoid or mitigate the flare-up of pain and/or inflammation; and provide the selected treatment to the treatment recipient.

Clause 19. A non-transitory computer-readable medium storing computer-executable instructions, the computer-executable instructions comprising at least one instruction for causing at least one processor to: determine, based on information associated with a treatment recipient, that a flare-up of pain and/or inflammation is in progress or that a likelihood of a flare-up of pain and/or inflammation exceeds a threshold level; and provide, to the treatment recipient, treatment to avoid or mitigate the flare-up of pain and/or inflammation, wherein the treatment comprises at least one of TENS therapy, PEMF therapy, cold laser therapy, IR therapy, red light therapy, heat therapy, or a combination thereof, sequentially, simultaneously, or a combination thereof.

Clause 20. The non-transitory computer-readable medium of clause 19, wherein the computer-executable instructions to provide treatment to the treatment recipient further comprise instructions causing the at least one processor to: notify the treatment recipient of the flare-up or the likelihood of a flare-up receive, from the treatment recipient, an indication of a selected treatment; and provide the selected treatment to the treatment recipient.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Further, those of skill in the art will appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the aspects disclosed herein may be implemented or performed with a general purpose processor, a DSP, an ASIC, an FPGA, or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The methods, sequences and/or algorithms described in connection with the aspects disclosed herein may be embodied directly in hardware, in a software module executed by a processor, or in a combination of the two. A software module may reside in random access memory (RAM), flash memory, read-only memory (ROM), erasable programmable ROM (EPROM), electrically erasable programmable ROM (EEPROM), registers, hard disk, a removable disk, a CD-ROM, or any other form of storage medium known in the art. An example storage medium is coupled to the processor such that the processor can read information from, and write information to, the storage medium. In the alternative, the storage medium may be integral to the processor. The processor and the storage medium may reside in an ASIC. The ASIC may reside in a treatment recipient terminal (e.g., UE). In the alternative, the processor and the storage medium may reside as discrete components in a user terminal.

In one or more example aspects, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

While the foregoing disclosure shows illustrative aspects of the disclosure, it should be noted that various changes and modifications could be made herein without departing from the scope of the disclosure as defined by the appended claims. For example, the functions, steps and/or actions of the method claims in accordance with the aspects of the disclosure described herein need not be performed in any particular order. Further, no component, function, action, or instruction described or claimed herein should be construed as critical or essential unless explicitly described as such. Furthermore, as used herein, the terms “set,” “group,” and the like are intended to include one or more of the stated elements. Also, as used herein, the terms “has,” “have,” “having,” “comprises,” “comprising,” “includes,” “including,” and the like does not preclude the presence of one or more additional elements (e.g., an element “having” A may also have B). Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”) or the alternatives are mutually exclusive (e.g., “one or more” should not be interpreted as “one and more”). Furthermore, although components, functions, actions, and instructions may be described or claimed in the singular, the plural is contemplated unless limitation to the singular is explicitly stated. Accordingly, as used herein, the articles “a,” “an,” “the,” and “said” are intended to include one or more of the stated elements. Additionally, as used herein, the terms “at least one” and “one or more” encompass “one” component, function, action, or instruction performing or capable of performing a described or claimed functionality and also “two or more” components, functions, actions, or instructions performing or capable of performing a described or claimed functionality in combination.

What is claimed is:

1. A method of treating pain and/or inflammation, the method comprising:
determining, based on information associated with a treatment recipient, that a flare-up of pain and/or inflammation is in progress or that a likelihood of a flare-up of pain and/or inflammation exceeds a threshold level; and
providing, to the treatment recipient, treatment to avoid or mitigate the flare-up of pain and/or inflammation.

2. The method of claim 1, wherein information associated with the treatment recipient comprises information about a current physical environment of the treatment recipient.

3. The method of claim 2, wherein the information about the current physical environment of the treatment recipient comprises temperature, humidity, barometric pressure, precipitation, time of day, day of week, season, month, day, year, or a combination thereof.

4. The method of claim 1, wherein information associated with the treatment recipient comprises information about a current or previous physical, mental, and/or emotional state of the treatment recipient.

5. The method of claim 4, wherein the information about the current or previous physical, mental, and/or emotional state of the treatment recipient comprises information indicating a level or location of pain, a level or location of inflammation, a level of hunger, a level of fatigue, a mood, or a combination thereof.

6. The method of claim 1, wherein determining, based on the information associated with the treatment recipient, that a flare-up of pain and/or inflammation is in progress or that a likelihood of a flare-up exceeds a threshold level comprises determining a flare-up and/or predicting a flare-up using a machine learning model that uses the information associated with the treatment recipient as an input.

7. The method of claim 1, wherein providing treatment to the treatment recipient comprises providing the treatment using a treatment unit comprising circuitry for providing transcutaneous electrical nerve stimulation (TENS), pulsed electromagnetic field (PEMF), cold laser emissions, infrared (IR) light emissions, red light emissions, and heat.

8. The method of claim 7, wherein providing treatment using the treatment unit comprises providing TENS therapy, PEMF therapy, cold laser therapy, IR therapy, red light therapy, heat therapy, or a combination thereof, sequentially, simultaneously, or a combination thereof.

9. The method of claim 1, wherein providing treatment to the treatment recipient comprises providing the treatment using a treatment unit comprising circuitry for providing pulsed electromagnetic field (PEMF), cold laser emissions, infrared (IR) light emissions, red light emissions, and heat.

10. The method of claim 9, wherein providing treatment using the treatment unit comprises providing PEMF therapy, cold laser therapy, IR therapy, red light therapy, heat therapy, or a combination thereof, sequentially, simultaneously, or a combination thereof.

11. The method of claim 1, wherein providing treatment to the treatment recipient comprises:
notifying the treatment recipient of the flare-up or the likelihood of a flare-up;
receiving, from the treatment recipient, an indication of a selected treatment to avoid or mitigate the flare-up of pain and/or inflammation; and
providing the selected treatment to the treatment recipient.

12. The method of claim 11, wherein notifying the treatment recipient of the flare-up or the likelihood of a flare-up comprises notifying the treatment recipient via a software application and wherein receiving, from the treatment recipient, the indication of the selected treatment comprises receiving the indication via the software application.

13. An apparatus for treating pain and/or inflammation, the apparatus comprising:
at least one memory;
at least one wireless transceiver coupled to at least one antenna;
at least one pulsed electromagnetic field (PEMF) induction coil;
at least one cold laser emitter;
at least one red light, infrared light, and/or heat (RIRH) emitter; and
processing circuitry communicatively coupled to the at least one memory, the at least one wireless transceiver, the at least one PEMF induction coil, the at least one cold laser emitter, and the at least one RIRH emitter,
wherein the processing circuitry is configured to provide a treatment to a treatment recipient, the treatment comprising at least one of PEMF therapy, cold laser therapy, infrared light therapy, red light therapy, heat therapy, or a combination thereof, sequentially, simultaneously, or a combination thereof.

14. The apparatus of claim 13, further comprising at least one pair of transcutaneous electrical nerve stimulation (TENS) electrodes communicatively coupled to the processing circuitry, wherein the processing circuitry is further configured to provide TENS therapy singly or in combination with other therapies, sequentially, simultaneously, or a combination thereof.

15. The apparatus of claim 13, wherein the processing circuitry is further configured to provide the treatment to the treatment recipient in response to instructions received via at least one of the wireless transceiver or an input device on or within the apparatus.

16. The apparatus of claim 13, wherein the processing circuitry is further configured to provide the treatment to the treatment recipient in response to determining, based on information associated with the treatment recipient, that a flare-up of pain and/or inflammation is in progress or that a likelihood of a flare-up exceeds a threshold level.

17. The apparatus of claim 16, wherein information associated with a treatment recipient comprises at least one of:
information about the treatment recipient's current or previous physical, mental, and/or emotional state; or
information about a current physical environment of the treatment recipient.

18. The apparatus of claim 16, wherein, to provide treatment to the treatment recipient, the processing circuitry is further configured to:
notify the treatment recipient of the flare-up or the likelihood of a flare-up;
receive, from the treatment recipient, an indication of a selected treatment to avoid or mitigate the flare-up of pain and/or inflammation; and
provide the selected treatment to the treatment recipient.

19. A non-transitory computer-readable medium storing computer-executable instructions, the computer-executable instructions comprising at least one instruction for causing at least one processor to:
determine, based on information associated with a treatment recipient, that a flare-up of pain and/or inflammation is in progress or that a likelihood of a flare-up of pain and/or inflammation exceeds a threshold level; and
provide, to the treatment recipient, treatment to avoid or mitigate the flare-up of pain and/or inflammation,
wherein the treatment comprises at least one of transcutaneous electrical nerve stimulation (TENS) therapy, pulsed electromagnetic field (PEMF) therapy, cold laser therapy, infrared (IR) therapy, red light therapy, heat therapy, or a combination thereof, sequentially, simultaneously, or a combination thereof.

20. The non-transitory computer-readable medium of claim 19, wherein the computer-executable instructions to provide treatment to the treatment recipient further comprise instructions causing the at least one processor to:
notify the treatment recipient of the flare-up or the likelihood of a flare-up
receive, from the treatment recipient, an indication of a selected treatment; and
provide the selected treatment to the treatment recipient.
